(19) United States
(12) Patent Application Publication
(10) Pub. No.: US 2004/0037895 A1
(43) Pub. Date: Feb. 26, 2004

(54) Methods of treating involuntary facial spasms and facial wrinkles

(76) Inventor: Alex Zhu, New York, NY (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/365,108

(22) Filed: Feb. 12, 2003

Related U.S. Application Data
(60) Provisional application No. 60/405,779, filed on Aug. 23, 2002.

(51) Int. Cl. A61K 33/06; A61K 31/704; A61K 31/58; A61K 31/4709

(52) U.S. Cl. 424/697; 514/37; 514/39; 514/176; 514/308

(57) ABSTRACT

The invention describes antibiotics, muscle relaxants and plant extracts that have neuromuscular blockade effects as well as methods of use thereof. These compounds can be used in the same clinical settings as botulinum toxin and may be used topically, thereby providing an advantage over botulinum toxin in terms of application and ease of use. The compounds can be used in pharmaceutical compositions for the treatment of involuntary muscle spasms and in cosmetic compositions for the treatment of facial wrinkles. Also provided are kits useful for therapeutic and/or cosmetic applications.

### RELATED APPLICATIONS

This application claims priority to U.S. Ser. No. 60/405,779, filed Aug. 23, 2002, which is incorporated herein by reference in its entirety.

### FIELD OF THE INVENTION

This invention relates generally to pharmaceutical compositions and methods for chemodenervation using compounds with botulinum toxin-like properties.

### BACKGROUND OF THE INVENTION

Several compounds are used to modulate the activity at neuromuscular junctions and display neuromuscular blockade effects. One such compound is botulinum toxin, which blocks the release of acetylcholine from the neuromuscular junction, and has been applied to a variety of therapeutic and cosmetic conditions. These applications include, but are not limited to, ocular disorders; dystonia, bleopharospasm, hemifacial spasm, synkinesis, and the involuntary facial muscle spasms caused by these disorders; gastrointestinal disorders; management of pain; and treatment of facial wrinkles. In all these cases, the toxin needs to be injected in the area where symptoms occur in order to exert a therapeutic effect.

The present invention can be utilized in a variety of therapeutic and cosmetic conditions where botulinum toxin is currently used (Binder et al. Dis Mon 48: 323-335, 2002; Epperson, Ala Med 65: 49-50, 1995; Khawaja et al. Int J Dermatol 40: 311-317, 2001; Mendez-Eastman, Plast Surg Nurs 20: 60-65, 2000; Verheyden and Blitzer, Dis Mon 48: 357-366, 2002). Additionally, synkinesis, a common phenomenon associated with facial paralysis, and facial wrinkles, is utilized as an example of a botulinum toxin-treatable condition to demonstrate novelty and utility of the invention.

Synkinesis

Approximately 7,000 neuron cell bodies make up the facial nerve, each of which innervates approximately 25 muscle fibers. The axons are surrounded by myelin, produced by the Schwann cells surrounding the axons. Three membranes comprise the nerve sheath. The epineurium is the outer covering, composed of loose areolar tissue, which separates the fascicles and holds them together. The perineurium is the next more inner layer. This is a dense layer of cells that are metabolically active and function as a diffusion barrier. The perineurium provides considerable strength to the nerve sheath. The individual nerve fibers are then each surrounded by the endoneurium.

Involuntary synkinesis between the orbicularis oculi (eye) and orbicularis oris (mouth) muscle is a very common side effect in patients recovering from facial nerve paralysis. It results from an aberrant connection of the motor fibers originally innervating orbicularis oris to the orbicularis oculi, causing lid closure whenever patients smile, talk or eat. If severe, this can lead to social embarrassment and functional visual loss.

When the facial nerve is damaged by surgery or viral infection, each of the nerve fibers needs to regrow. However, the appropriate nerve fiber ends do not always grow and connect correctly, resulting in a “crossed wires” phenomenon. This results in the misdirection of nerve impulses at the site of the nerve injury. Synkinesis varies in severity from mild to severe. In its worst form, it can result in uncontrollable movement of the facial muscles on the affected side during any attempted expression. The affected side of the face may feel tight as the result of the uncontrolled muscle contractions (spasms).

Current Medical Treatment

Medical Therapy:

Currently, the only medical treatment for synkinesis is the injection of botulinum toxin, currently manufactured and marketed by Allergan, Inc. under the brand name Botox (Armstrong et al. Clin Otolaryngol 21: 15-20,1 1996; Laskawi et al. Eur Arch Otorhinolaryngol S195-S199, 1994; Mountain et al. Clin Otolaryngol 17: 223-224, 1992; Roggenkamper et al. Doc Ophthalmol 86: 395-402, 1994). The use of botulinum toxin as chemical neurectomy offers an effective though not a curative treatment. The toxin is injected subcutaneously in the pretarsal areas in both the upper and lower lids as in blepharospasm. This has the effect of paralyzing the orbicularis and therefore reduces the synkinesis. The treatment takes a few days to work and usually lasts between two to three months. Repeated injection is required when the effect wanes.

Facial muscles have a tendency to become hypertonic (overactive) after paralysis. Weakening or re-paralyzing the muscles with Botox injection may temporarily ease the effects of some synkinesis and hypertonic muscles. Spasms can be reduced along with the pain and discomfort associated with them.

Some patients may benefit from a special form of physical therapy called facial retraining (Diels and Combs, Otolaryngol Clin North Am 30: 727-743, 1997).

Electrical stimulation has been discouraged by most doctors based on mounting evidence that it may be harmful to the nerve's ability to regenerate. Electrical stimulation may result in a mass contraction of the facial muscles thereby producing an undesirable, uncoordinated muscle response.

Surgical Therapy:

Surgical repair of the facial nerve is generally performed in cases of complete paralysis. Presently, three options exist for repair of the facial nerve. Options include direct repair, cable nerve grafting, and nerve substitution techniques (Angeli and Chiossone, Otolaryngol Clin North Am 30: 683-700, 1997). Synkinesis is expected in all cases, regardless of the mode of repair chosen. The best result one can hope for is a House-Brackman grade III. The House-Brackmann grading scale (6 levels) for facial paralysis is used to objectively describe the degree of facial paralysis (House and Brackman, Otolaryngol Head Neck Surg. 93,146-147, 1985).

| Grade | Characteristics |
|---|---|
| I. Normal | Normal facial function in all areas |
| II. Mild dysfunction | Gross |
| | Slight weakness noticeable on close |
| | inspection |
| | May have slight synkinesis |
| | At rest, normal symmetry and tone |
| | Motion |
| | Forehead - Moderate-to-good function |
| | Eye - Complete closure with minimal |
| | effort |
| | Mouth - Slight asymmetry |
| III. Moderate | Gross |
| dysfunction | Obvious but not disfiguring difference |
| | between the two sides |
| | Noticeable but not severe synkinesis, |
| | contracture, or hemifacial spasm |
| | At rest, normal symmetry and tone |
| | Motion |
| | Forehead - Slight-to-moderate movement |
| | Eye - Complete closure with effort |
| | Mouth - Slightly weak with maximum |
| | effort |
| IV. Moderately severe | Gross, Obvious weakness and/or |
| dysfunction | disfiguring asymmetry |
| | At rest, normal symmetry and tone |
| | Motion |
| | Forehead - None |
| | Eye - Incomplete closure |
| | Mouth - Asymmetric with maximum effort |
| V. Severe dysfunction | Gross |
| | Only barely perceptible motion |
| | At rest, asymmetry |
| | Motion |
| | Forehead - None |
| | Eye - Incomplete closure |
| | Mouth - Slight movement |
| VI. Total paralysis | No movement |

Donor site morbidity is also expected for the hypoglossal crossover technique (tongue weakness), great auricular harvest (ear numbness), and sural nerve harvest (lateral leg numbness). Complications for each of the procedures can include hematoma and infection.

Facial Wrinkles

There are two different types of wrinkles, fine wrinkles and dynamic wrinkles. Traditionally, fine wrinkles are believed to result from aging, sun exposure, and reduction in collagen and elastic fibers of the skin. Deep wrinkles are associated with the build-up of the musculature below the skin surface. When people with healthy skin are making facial expressions, they show dynamic wrinkles around the facial expression muscles. When those muscles are at rest, skin resumes its most smoothness due to elasticity. It is believed, however, that muscles at resting stage are in a slight contractile posture due to base-level / spontaneous quantal release of acetylcholine. Healthy skin looks smooth because elasticity overcomes this mild contractile muscle posture to hide obvious wrinkles. Facial wrinkles are generated presumably by the breakdown of skin support of collagen and elastin fiber due to aging and sun exposure, diminished function of sweat glands and fat pad atrophy beneath the dermis. In addition, years of repeating the facial muscle contraction can thicken the muscle layer and thus deepen the wrinkles.

Current Facial Wrinkle Treatments

Many approaches are taken to reduce the appearance of facial wrinkles based on the understanding of the molecular basis of wrinkle formation. Such treatments include cosmetic products, drug therapy and surgical procedures. For example, many cosmetic products contain alpha hydroxy acids, which may stimulate collagen synthesis. Another common treatment utilizes retinol, a derivative of vitamin A, (or its stronger, prescribed version Retin-A and Renova) which helps collagen production. Antioxidants such as vitamin C and E and coenzyme Q-10 are believed to counteract free radicals, which damage cells and cause aging and have been used in treatments of wrinkles. Recently, the FDA approved cosmetic use of Botox (an extremely diluted form of botulinum toxin) to treat glabella frown lines.

Botulinum Toxin Therapy

Botulinum toxin blocks the release of acetylcholine from the neuromuscular junction, and has been applied to a variety of cosmetic conditions (Binder et al. Dis Mon 48: 323-335, 2002; Epperson, Ala Med 65: 49-50, 1995; Khawaja et al. Int J Dermatol 40: 311-317, 2001; Mendez-Eastman, Plast Surg Nurs 20: 60-65, 2000; Verheyden and Blitzer, Dis Mon 48: 357-366, 2002). These applications include treatment of facial wrinkles. In such cases, the toxin needs to be injected in the area where symptoms occur in order to exert an effect.

Cosmetic use of botulinum toxin in minimizing facial wrinkles underscores the theory that contractile muscle fiber, which is under the direct control of the neural motor influx, is likely to play an important role in the formation of wrinkles, particularly glabellar frown lines. Locally blocking the motor contraction by botulinum toxin not only minimizes wrinkles but also exerts a “smoothing” effect on the skin's microrelief.

Disadvantages of Botulinum Toxin Therapy

Botulinum toxin, produced by Clostridium botulinum, consists of a 100-kD heavy chain linked to a 50-kD light chain by a disulfide bond (Singh, Nat Struct Biol 7: 617-619, 2000). The toxin blocks the release of acetylcholine from cholinergic nerve endings by a three-step mechanism. First, the toxin binds to presynaptic terminals. Second, the toxin is internalized by receptor-mediated endocytosis and released into the cytosol. Finally, the light chain enzymatically blocks exocytosis (release) of acetylcholine at the neuromuscular junction.

Complications associated with the botulinum toxin therapy may partially result from the apparent diffusion of the toxin from the injected muscle(s) to adjacent muscles (Khawaja et al. Int J Dermatol 40: 311-317, 2001). When such diffusion occurs, the resulting muscle paralysis can cause double vision or ptosis so severe that sight is obstructed by the drooping eyelid.

It was initially believed, based on the experience with food-borne botulism, that individuals exposed to botulinum toxin did not produce antibodies against the toxin, due to extremely low dosage of the toxin therapeutically used. However, a different picture has emerged from the toxin therapy. It has been observed that some patients, who initially benefited from the toxin, later became insensitive (refractory, resistant) to its use. This insensitivity has been attributed to the development, upon repeated injections with the toxin, of antibodies against the toxin. Jankovic and Schwartz found neutralizing antibodies against the toxin in 5 (37.5%) sera from 14 patients characterized as “non-responders” to botulinum toxin therapy, whereas no antibodies were found in 32 patients characterized as “responders” to the toxin (P<0.0001) (Jankovic and Schwartz, Arch Neurol 48: 1253-1256, 1991). In a group of 20 patients categorized as “maintained response” or as “diminished response” who had been treated for several years with botulinum type A toxin, seven (35%) of the patients were found to have toxin-neutralizing antibodies (Hambleton et al. BMJ 304: 959-960, 1992).

Therefore, long-term use of botulinum toxin, even at very low concentration, is likely to induce toxin-neutralizing antibodies, which will decrease the therapeutic effect. For the same reason, people who are vaccinated against botulinum toxin for preventing botulism cannot benefit from the toxin therapy even if such a medical need arises.

Another inherent drawback for botulinum toxin treatment is that the toxin has to be delivered to the symptomatic site by subcutaneous injection. As a protein of 150 kDa, the toxin is far too large a molecule to penetrate the skin to exert its therapeutic effect.

### SUMMARY OF THE INVENTION

In one aspect, the invention disclosed herein includes a method for localized chemodenervation by topically administering a pharmaceutical composition containing a pharmaceutically effective amount of an antibiotic, aminoglycoside antibiotic, polymyxin B, tetracycline, lincosamide or muscle relaxant and a pharmaceutically acceptable carrier.

In another aspect, the invention includes a method for localized chemodenervation by topically administering a pharmaceutical composition containing a pharmaceutically effective amount of one or more compounds selected from the group consisting of a magnesium salt, an organic magnesium compound or a plant extract, and a pharmaceutically acceptable carrier.

In yet another aspect, the invention includes a method for treating involuntary facial muscle spasms by topically administering a pharmaceutical composition containing a pharmaceutically effective amount of an antibiotic, aminoglycoside antibiotic, polymyxin B, tetracycline, lincosamide or muscle relaxant and a pharmaceutically acceptable carrier.

In another aspect, the invention includes a method for treating involuntary facial muscle spasms by topically administering a pharmaceutical composition containing a pharmaceutically effective amount of one or more compounds selected from the group consisting of a magnesium salt, an organic magnesium compound or a plant extract, and a pharmaceutically acceptable carrier.

In yet another aspect, the invention includes a method for reducing facial wrinkles by topically administering a pharmaceutical composition containing a pharmaceutically effective amount of an antibiotic, aminoglycoside antibiotic, polymyxin B, tetracycline, lincosamide or muscle relaxant and a pharmaceutically acceptable carrier.

In another aspect, the invention includes a method for reducing facial wrinkles by topically administering a pharmaceutical composition containing a pharmaceutically effective amount of one or more compounds selected from the group consisting of a magnesium salt, an organic magnesium compound or a plant extract, and a pharmaceutically acceptable carrier.

In yet another aspect, the invention includes a pharmaceutical composition for topical administration for localized chemodenervation containing pharmaceutically effective amount of an antibiotic, aminoglycoside antibiotic, polymyxin B, tetracycline, lincosamide or muscle relaxant in a pharmaceutically acceptable medium compatible with human skin. In a preferred aspect, the pharmaceutical composition further contains a magnesium salt or organic magnesium compound.

In yet another aspect, the invention includes a pharmaceutical composition for topical administration for treating involuntary facial muscle spasms containing effective amount of an antibiotic, aminoglycoside antibiotic, polymyxin B, tetracycline, lincosamide or muscle relaxant in a medium compatible with human skin. In a preferred aspect, the pharmaceutical composition further contains a magnesium salt or organic magnesium compound.

In yet another aspect, the invention includes a pharmaceutical composition for topical administration for reducing facial wrinkles containing effective amount of an antibiotic, aminoglycoside antibiotic, polymyxin B, tetracycline, lincosamide or muscle relaxant in a medium compatible with human skin. In a preferred aspect, the pharmaceutical composition further contains a magnesium salt or organic magnesium compound.

In a further aspect, the invention includes a kit containing a pharmaceutical composition for localized chemodenervation containing a pharmaceutically effective amount of an antibiotic, aminoglycoside antibiotic, polymyxin B, tetracycline, lincosamide, muscle relaxant, or plant extract and a pharmaceutically acceptable carrier, and instructions for topical administration of the composition.

In yet another aspect, the invention includes a kit containing a pharmaceutical composition for treating involuntary facial muscle spasms containing a pharmaceutically effective amount of an antibiotic, aminoglycoside antibiotic, polymyxin B, tetracycline, lincosamide, muscle relaxant, or plant extract and a pharmaceutically acceptable carrier, and instructions for topical administration of the composition.

In yet another aspect, the invention includes a kit containing a pharmaceutical composition for reducing facial wrinkles containing a pharmaceutically effective amount of an antibiotic, aminoglycoside antibiotic, polymyxin B, tetracycline, lincosamide, muscle relaxant, or plant extract and a pharmaceutically acceptable carrier, and instructions for topical administration of the composition.

The aminoglycoside antibiotic can be amikacin, gentamicin, kanamycin, neomycin, netilmicin, paromomycin, streptomycin and tobramycin. The amount of neomycin can be between about 2.5 mg/g and about 250 mg/g. The lincosamide can be clindamycin or lincomycin. The muscle relaxant can be a non-depolarizing agent or a depolarizing agent. The non-depolarizing agent can be pancuronium, vecuronium, mivacurium or rocuronium. The depolarizing agent can be succinylcholine or decamethonium. The muscle relaxant can be a magnesium salt or an organic magnesium compound. The plant extract can be toosendanin, coryneine, banana truck extract or curare. The amount of polymyxin B can be between about 800 U/g and about 80,000 U/g.

The pharmaceutical composition can further contain a magnesium salt or an organic magnesium compound. The magnesium salt can be magnesium sulfate. The amount of magnesium sulfate can be between about 2 mg/g and about 200 mg/g.

The involuntary facial muscle spasms can be caused by synkinesis, ocular disorders, dystonia, hemifacial spasm, or blepharospasm.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a photograph showing the effect of topical cream on glabellar frown lines

FIG. 2 is a schematic representation illustrating a mouse model for Botox-like compounds and their antagonists.

FIG. 3 is a bar graph showing the effect of topical cream on Botox-induced symptoms in 7 mice.

FIG. 4 is a bar graph showing the effect of topical cream on Botox-induced symptoms in 6 mice.

### DETAILED DESCRIPTION OF THE INVENTION

Disclosed herein are compounds that also have neuromuscular blockade effects, which can be useful in the same clinical settings as botulinum toxin. These compounds may be used topically, providing an advantage over botulinum toxin in application and ease of use. Also disclosed are formulations of two topical creams comprising these compounds that are similar to botulinum toxin in their mechanism of action and are effective in both therapeutic and cosmetic settings.

The present invention is based in part on classes of compounds that are functionally similar to botulinum toxin in terms of blocking neuromuscular transmission, but are able to exert physiological effects by topical administration. Additionally, the invention provides pharmaceutical compositions, kits, and methods of localized chemodenervation and methods of treating involuntary facial muscle spasms using these compounds.

As used herein, the terms “localized chemodenervation” and “localized denervation” mean denervation of a particular anatomical region, usually a muscle or group of anatomically- and/or physiologically-related muscles, which results from administration of a chemodenervating agent, for example a neurotoxin, to the particular anatomical region.

The term “treating” in its various grammatical forms in relation to the present invention refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. The term “reducing” in its various grammatical forms in relation to the present invention refers to lessening, curing, reversing, attenuating, alleviating, minimizing, suppressing or halting the deleterious effects of a disease state, disease progression, disease causative agent (e.g., bacteria or viruses) or other abnormal condition.

Any compounds disclosed herein shall also include all derivatives and analogs.

Aminoglycoside Antibiotics

In one aspect, the invention includes aminoglycoside antibiotics used for localized chemodenervation, treatment of involuntary facial muscle spasms and reducing facial wrinkles. Aminoglycoside antibiotics are commonly used to treat certain bacterial infections. Preferred aminoglycoside antibiotics include but are not limited to amikacin, gentamicin, kanamycin, neomycin, netilmicin, paromomycin, streptomycin, and tobramycin. All of these antibiotics have the similar base chemical structure. As used herein, the term “antibiotic” or “antibiotics” means a chemical substance produced by a microorganism which has the capacity, in dilute solutions, to inhibit the growth of or to kill other microorganisms.

Neuromuscular blockade is a well-known adverse effect that follows the administration of aminoglycoside antibiotics (Corrado et al. Acta Physiol Pharmacol Latinoam 39: 419-430, 1989; Suarez-Kurtz, Acta Physiol Pharmacol Latinoam 39: 407-418, 1989). Many animal studies and clinical case reports have shown that aminoglycoside antibiotics inhibit acetylcholine release at the motor nerve terminal (presynaptic) and/or reducing response to acetylcholine (postsynaptic) (Fiekers, Acta Physiol Pharmacol Ther Latinoam 49: 242-250, 1999).

Aminoglycosides are absorbed very poorly from the gastrointestinal tract. Typical routes of administration are by intramuscular injection, intravenous injection, topical application, or inhalation. Time of peak concentrations occurs between 30 to 120 minutes after intramuscular injection. Intravenous infusion is the preferred administration route for most aminoglycosides. The drugs are diluted in 25 to 100 ml of dextrose or saline, and infused over 30 to 60 minutes.

Tetracyclines

In another aspect, the invention includes tetracyclines used for localized chemodenervation, treatment of involuntary facial muscle spasms and reducing facial wrinkles. Tetracyclines have a modest effect on the release of acetylcholine (Pittinger and Adamson, Annu Rev Pharmacol 12: 169-184, 1972; Wright and Collier, Can J Physiol Pharmacol 54: 926-936, 1976). This effect is partially reversed by calcium, but not by neostigmine. In addition, this effect can be antagonized by 4-aminopyridine, suggesting that its mode of action is on presynaptic calcium ion flux.

Polymyxin B

In another aspect, the invention includes polymyxin B used for localized chemodenervation, treatment of involuntary facial muscle spasms and reducing facial wrinkles. Polymyxin B is a complex mixture of closely related polypeptides isolated from various strains of Bacillus Polymyxa and is active against a wide spectrum of Gram-negative bacteria (Govaerts et al. J Pept Sci 8: 45-55, 2002: Orwa et al. J Chromatogr A 912: 369-373, 2001). Approved as topical antibiotic for over 50 years, polymyxin B is also administered orally for GI decontamination prior to abdominal surgery and intrathecally for the treatment of bacterial meningitis. Polymyxin B is widely used as topical antibiotics with the concentration up to 10,000 U per gram (over-the-counter drug).

Polymyxin B produces neuromuscular blockade by acting at pre- and postsynaptic sites (Durant and Lambert, Br J Pharmacol 72: 41-47, 1981). Its postsynaptic effect is believed to be the main cause of the neuromuscular blockade and difficult to reverse by calcium. Of all the antibiotics, polymyxin B is considered to be the most potent neuromuscular blocking agent.

Lincosamides

In another aspect, the invention includes lincosamides used for localized chemodenervation, treatment of involuntary facial muscle spasms and reducing facial wrinkles. The pre- and postjunctional effects of the lincosamide antibiotics, clindamycin and lincomycin, were studied by Fiekers et al (J Pharmacol Exp Ther 227: 308-315, 1983) in voltage-clamped transected twitch fibers of costocutaneous muscles of garter snakes (species Thamnophis). Their results suggest that the neuromuscular blocking effects of clindamycin involve both pre-and postjunctional sites, whereas the effects of lincomycin are primarily on the postjunctional receptor-channel complex (Fiekers et al. J Pharmacol Exp Ther 227: 308-315, 1983; Wright and Collier, Can J Physiol Pharmacol 54: 937-944, 1976).

Magnesium

In another aspect, the invention includes magnesium or an organic magnesium compound used for localized chemodenervation, treatment of involuntary facial muscle spasms and reducing facial wrinkles. Magnesium is one of the most common cations in the body and plays a fundamental role as a co-factor in more than 300 enzymatic reactions in most metabolisms (Fawcett et al. Br J Anaesth 83: 302-320, 1999). For instance, magnesium is required for the transfer of phosphate groups in signal transduction pathways, ATP involved reactions, replication and transcription of DNA and the translation of mRNA. In addition, magnesium is involved in membrane stability, nerve conduction, iron transport, and calcium-channel activity.

Magnesium deficiency, caused by physiological and dietary conditions, is common and usually multifactorial. Epidemiological studies trace the prevalence of cardiovascular disease and cardiac deaths to the degree of magnesium depletion due to a diet and drinking water low in magnesium.

High magnesium concentration inhibits release acetylcholine from the presynaptic nerve terminal, which can be reversed by calcium. Magnesium sulfate reduces striated muscle contractions and blocks peripheral neuromuscular transmission by reducing acetylcholine release at the myoneural junction. In emergency care, magnesium sulfate is used in the management of seizures associated with toxemia of pregnancy and in uterine relaxation (to inhibit contractions of premature labor).

Many different magnesium salts are used in clinical setting, including magnesium oxide, mineral magnesium salts (e.g., sulfate, nitrate, chloride and carbonate) and organic magnesium (e.g., ascorbate, acetate, bicitrate, methionate, levulinate, glycero-phosphate, gluconate, aspartate, propionate, lactate, fumarate, glutamate and pyrrolidone-carboxylate).

Plant and herb extracts

In another aspect, the invention includes plant and herb extracts used for localized chemodenervation, treatment of involuntary facial muscle spasms and reducing facial wrinkles. As used herein, the term “plant extract” means compounds derived from plants, herbs or botanicals. Preferred plant extracts include but are not limited to toosendanin, coryneine, banana truck extract or curare.

Toosendanin is a triterpenoid derivative of molecular weight of 574 (Ding et al. Neurosci Res 41: 243-249, 2001; Wang and Shi, Neurosci Res 40: 211-215, 2001; Xu and Shi, Brain Res 631: 46-50, 1993). As an active compound in Fructus Maliae Toosendan or Cortex Maliae, and in Chinese traditional medicine, toosendanin is reported to be a neuromuscular blocker acting on the presynaptic terminals. The blocking action of toosendanin is preceded by a facilitating phase, which may last for several days. It is interesting that during such a facilitating phase, the tolerance of the neuromuscular junction towards botulinum toxin is enhanced significantly.

Aconite root is commonly used in Chinese and Japanese herbal medicine for relieving muscle pain. Isolated from aconite roots, coryneine is a quaternary ammonium derivative of dopamine, which exhibits a depolarizing neuromuscular blocking action (Nojima et al. J Asian Nat Prod Res 2: 195-203, 2000). Kimura et al showed that coryneine at 20-150 uM blocked the nerve-evoked twitch response, which was reversed by neostigmine, a cholinesterase inhibitor (Kimura et al. Biol Pharm Bull 18: 691-695, 1995).

It was reported that the juice of the banana truck induces a muscle paralyzing effect in both directly and indirectly stimulated preparations (Singh et al. Arch Int Pharmacodyn Ther324: 105-113, 1993; Singh and Dryden, Toxicon 23: 973-981, 1985). The neuromuscular blockade was reversed by calcium, but only when added before complete paralysis of the muscle. A chemical composition study of banana truck revealed that the active component responsible for muscle paralysis mainly consisted of monopotassium ovalate. However, another study claimed that two major active principles causing muscle paralysis are potassium nitrate and magnesium nitrate.

An extract from the bark and stems of curare (Chondodendron tomentosum) is the source of a potent isoquinoline alkaloid used in the deadly poison curare. Amazonian Indians use the gummy extract to coat the poison darts of their blowguns. The alkaloid D-tubocurarine blocks acetylcholine receptor sites at neuromuscular junctions, causing relaxation and paralysis of muscles, including respiratory organs and the heart (Mallart and Molgo, J Physiol 276: 343-352, 1978; Manalis, Nature 267: 366-368, 1977). D-tubocurarine has been used to relax the heart muscle during open-heart surgery. It has also been used to treat the spastic paralysis of tetanus toxin from the bacterium Clostridium botulinum.

Other plants or herbs that have demonstrated neuromuscular blocking activity include, but are not limited to, piper methysticum (Singh, J Ethnopharmacol 7: 267-276, 1983), Atractylodes lancea (Kimura et al. Neuropharmacology 30: 835-841, 1991), passiflora incarnata, ciminifuga racemose, camellia sinensis (Das et al. J Ethnopharnacol 57: 197-201, 1997), sarcolobus globosus (Mustafa, Toxicon 31: 67-74, 1993), ipomoea fistulosa (Abdelhadi et al. Clin Exp Pharmacol Physiol 13: 169-171, 1986), piper sarmentosum (Ridtitid et al. J Ethnopharnacol 61: 135-142, 1998), Saccharum officinarum and Psidium guajava (Re et al. Pharmacol Res 39: 239-245, 1999).

Muscle Relaxants

In another aspect, the invention includes muscle relaxants used for localized chemodenervation, treatment of involuntary facial muscle spasms and reducing facial wrinkles. Studies of nerve-impulse transmission at neuromuscular junctions have indicated that acetylcholine released from the presynaptic site binds to specific receptors on postsynaptic membrane of motor end plate. Such a binding produces a large change in ion permeability properties (depolarizing effect) of the postsynaptic membrane.

The transmission of acetylcholine at the neuromuscular joints can be blocked by either non-depolarizing agents or depolarizing agents. The non-depolarizing agents compete with acetylcholine for the postsynaptic receptor site, thus preventing depolarization. Preferred non-depolarizing agents include, but are not limited to, pancuronium, vecuronium, mivacurium, or rocuronium. The depolarizing blocking agents act like an excess of acetylcholine to depolarize the muscle receptor site and prevent its repolarization. Thus, there is an initial depolarization resulting in muscle contraction. Since the muscle receptor site cannot depolarize, complete skeletal muscle relaxation follows. Preferred depolarizing agents include, but are not limited to, succinylcholine or decamethonium.

Most of those muscle relaxants described above offer rapid onset (several minutes) and short duration (up to two hours), and have been extensively used as anesthesia in surgical procedures.

Although the neuromuscular blocking properties of the above said compounds have been reported in the literature, their clinical utilities have not been fully explored, except for the muscle relaxants and magnesium used as anesthesia in surgical procedures and other medical conditions. In fact, neuromuscular blockade is considered as the undesirable side effect for the antibiotics mentioned above. Furthermore, none of the above said compounds, with exception for polymyxin B, has been topically used in clinical settings.

Methods for Localized Chemodenervation

Aminoglycoside Antibiotics:

The present invention includes methods for localized chemodenervation. In one aspect, the present invention includes a method for localized chemodenervation by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic and a pharmaceutically acceptable carrier. The aminoglycoside antibiotic can be amikacin, gentamicin, kanamycin, neomycin, netilmicin, paromomycin, streptomycin or tobramycin. The pharmaceutical composition can further comprise a magnesium salt or an organic magnesium compound.

Tetracyclines:

The invention also includes a method for localized chemodenervation by topically administering a pharmaceutical composition for comprising a pharmaceutically effective amount of tetracycline and a pharmaceutically acceptable carrier. In a preferred embodiment, the pharmaceutical composition can further comprise a magnesium salt or an organic magnesium compound.

Polymyxin B:

In another aspect, the invention includes a method for localized chemodenervation by topically administering a pharmaceutical composition comprising a pharmaceutically effective amount of polymyxin B and a pharmaceutically acceptable carrier. In a preferred embodiment, the pharmaceutical composition can further comprise a magnesium salt or an organic magnesium compound.

Lincosamides:

The invention also includes a method for localized chemodenervation by topically administering a pharmaceutical composition comprising a pharmaceutically effective amount of lincosamide and a pharmaceutically acceptable carrier. The lincosamide can be clindamycin or lincomycin. In a preferred embodiment, the pharmaceutical composition can further comprise a magnesium salt or an organic magnesium compound.

Muscle Relaxants:

In another aspect, the invention includes a method for localized chemodenervation by topically administering a pharmaceutical composition comprising a pharmaceutically effective amount of a muscle relaxant and pharmaceutically acceptable carrier. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound.

Plant/Herb Extracts:

The invention also includes a method for localized chemodenervation by topically administering a pharmaceutical composition comprising a pharmaceutically effective amount of one or more compounds selected from the group consisting of a magnesium salt, an organic magnesium compound or a plant extract, and a pharmaceutically acceptable carrier. The plant extract can be toosendanin, coryneine, banana truck extract or curare.

Methods for Treating Involuntary Facial Muscle Spasms

Aminoglycoside Antibiotics:

The present invention includes methods for treating involuntary facial muscle spasms. The involuntary facial muscle spasms can be caused by synkinesis, ocular disorders, dystonia, hemifacial spasm, or blepharospasm. In one aspect, the present invention includes a method for treating involuntary facial muscle spasms by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic and a pharmaceutically acceptable carrier. The aminoglycoside antibiotic can be amikacin, gentamicin, kanamycin, neomycin, netilmicin, paromomycin, streptomycin or tobramycin. The pharmaceutical composition can further comprise a magnesium salt or an organic magnesium compound.

Tetracyclines:

The invention also includes a method for treating involuntary facial muscle spasms by topically administering a pharmaceutical composition comprising a pharmaceutically effective amount of tetracycline and a pharmaceutically acceptable carrier. In a preferred embodiment, the pharmaceutical composition can further comprise a magnesium salt or an organic magnesium compound.

Polymyxin B:

In another aspect, the invention includes a method for treating involuntary facial muscle spasms by topically administering a pharmaceutical composition comprising a pharmaceutically effective amount of polymyxin B and a pharmaceutically acceptable carrier. In a preferred embodiment, the pharmaceutical composition can further comprise a magnesium salt or an organic magnesium compound.

Lincosamides:

The invention also includes a method for treating involuntary facial muscle spasms by topically administering a pharmaceutical composition comprising a pharmaceutically effective amount of lincosamide and a pharmaceutically acceptable carrier. The lincosamide can be clindamycin or lincomycin. In a preferred embodiment, the pharmaceutical composition can further comprise a magnesium salt or an organic magnesium compound.

Muscle Relaxants:

In another aspect, the invention includes a method for treating involuntary facial muscle spasms by topically administering a pharmaceutical composition comprising a pharmaceutically effective amount of a muscle relaxant and pharmaceutically acceptable carrier. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound.

Plant/Herb Extracts:

The invention also includes a method for treating involuntary facial muscle spasms by topically administering a pharmaceutical composition comprising a pharmaceutically effective amount of one or more compounds selected from the group consisting of a magnesium salt, an organic magnesium compound or a plant extract, and a pharmaceutically acceptable carrier. The plant extract can be toosendanin, coryneine, banana truck extract or curare.

Methods for Reducing Facial Wrinkles

Aminoglycoside Antibiotics:

The present invention includes methods for reducing facial wrinkles. In one aspect, the present invention includes a method for reducing facial wrinkles by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic and a pharmaceutically acceptable carrier. The aminoglycoside antibiotic can be amikacin, gentamicin, kanamycin, neomycin, netilmicin, paromomycin, streptomycin or tobramycin. The pharmaceutical composition can further comprise a magnesium salt or an organic magnesium compound.

In another preferred embodiment, the pharmaceutical composition for topical administration for reducing facial wrinkles comprises effective amounts of an aminoglycoside antibiotic and a magnesium salt or organic magnesium compound in a medium compatible with human skin. The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is a pharmaceutical composition comprising Cream II (described below).

Tetracyclines:

The invention also includes a method for reducing facial wrinkles by topically administering a pharmaceutical composition comprising a pharmaceutically effective amount of tetracycline and a pharmaceutically acceptable carrier. In a preferred embodiment, the pharmaceutical composition can further comprise a magnesium salt or an organic magnesium compound.

Polymyxin B:

In another aspect, the invention includes a method for reducing facial wrinkles by topically administering a pharmaceutical composition comprising a pharmaceutically effective amount of polymyxin B and a pharmaceutically acceptable carrier. In a preferred embodiment, the pharmaceutical composition can further comprise a magnesium salt or an organic magnesium compound.

In another preferred aspect, the pharmaceutical composition for topical administration for reducing facial wrinkles comprises effective amounts of polymyxin B and a magnesium salt or organic magnesium compound in a medium compatible with human skin. The magnesium salt can be magnesium sulfate. In preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. The most preferred embodiment is a pharmaceutical composition comprising Cream I (described below).

Lincosamides:

The invention also includes a method for reducing facial wrinkles by topically administering a pharmaceutical composition comprising a pharmaceutically effective amount of lincosamide and a pharmaceutically acceptable carrier. The lincosamide can be clindamycin or lincomycin. In a preferred embodiment, the pharmaceutical composition can further comprise a magnesium salt or an organic magnesium compound.

Muscle Relaxants:

In another aspect, the invention includes a method for reducing facial wrinkles by topically administering a pharmaceutical composition comprising a pharmaceutically effective amount of a muscle relaxant and pharmaceutically acceptable carrier. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound.

Plant/Herb Extracts:

The invention also includes a method for reducing facial wrinkles by topically administering a pharmaceutical composition comprising a pharmaceutically effective amount of one or more compounds selected from the group consisting of a magnesium salt, an organic magnesium compound or a plant extract, and a pharmaceutically acceptable carrier. The plant extract can be toosendanin, coryneine, banana truck extract or curare.

Pharmaceutical Compositions

The compounds, e.g., antibiotics (including but not limited to: aminoglycoside antibiotics, tetracyclines, polymyxin B, and lincosamides), magnesium and muscle relaxants (also referred to herein as “active compounds”) of the invention, and derivatives, fragments, analogs and homologs thereof, can be incorporated into pharmaceutical compositions suitable for administration. Such compositions typically comprise the active compounds and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Transdermal or Transmucosal Administration:

Systemic administration can also be by transmucosal or transdermal means. In a preferred embodiment, the pharmaceutical compositions of the present invention are administered topically. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment of the present invention, for topical application, the compositions of the invention comprise a medium (vehicle, diluent or carrier), which is compatible with human skin. These compositions can be, in particular, in the form of aqueous, alcoholic or aqueous/alcoholic solutions, ointments, lotions, gels, water-in-oil or oil-in-water emulsions having the appearance of a cream or a gel, microemulsions or aerosols, or alternatively in the form of vesicular dispersions containing ionic and/or nonionic lipids. These pharmaceutical dosage units are formulated according to the usual techniques in the fields under consideration.

In known fashion, the compositions of the invention can also contain adjuvants and additives that are common in the corresponding fields, such as hydrophilic or lipophilic gelling agents, preservatives, antioxidants, solvents, fragrances, fillers, UV-screening agents and dyestuffs and colorants. Moreover, these compositions can contain hydrophilic or lipophilic active agents. The amounts of these various adjuvants, additives or active agents are those used conventionally in the cosmetic or pharmaceutical field, and, for example, constitute from 0.01% to 5% of the total weight of the composition. Depending on their nature, these adjuvants or active agents can be introduced into the fatty phase, into the aqueous phase and/or into lipid vesicles.

In order to deliver the said compounds to the deep layers of the skin, the mixture can also be incorporated into the aqueous chambers of liposomes commercially prepared from phosphatidylcholine (lecithin). Liposomes are used widely because of their ability of penetrating deeply into the skin. In addition, applying controlled-release delivery technology known to the industry, function ingredients can be delivered at a pre-determined rate to achieve long-lasting therapeutic effects.

It is especially advantageous to formulate topically administered pharmaceutical compositions in a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. The appropriate dosage or dose is often referred to as an effective amount. As used herein, the terms “effective amount” and “pharmaceutically effective amount” mean the ability to denervate or treat or reduce a disease or a disorder or a symptom thereof.

Polymyxin B Pharmaceutical Compositions:

In one aspect, the invention includes a pharmaceutical composition for topical administration for localized chemodenervation comprising pharmaceutically effective amounts of polymyxin B and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of polymyxin B and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of polymyxin B and a magnesium salt or organic magnesium compound in a pharmaceutically acceptable medium compatible with human skin.

The magnesium salt can be magnesium sulfate. In preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. The most preferred embodiment is Cream I which is a pharmaceutical composition comprising distilled water, polymyxin B, magnesium sulfate, glycerine, glyceryl dilaurate, glyceryl alcohol, ceteareth-20, isopropyl myristate, imidurea NF, guar gum, methyl pareben, and propyl paraben.

Aminoglycoside Antibiotic Pharmaceutical Compositions:

The invention also includes a pharmaceutical composition for topical administration for localized chemodenervation comprising pharmaceutically effective amounts of an aminoglycoside antibiotic and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of aminoglycoside antibiotic and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of aminoglycoside antibiotic and a magnesium salt or organic magnesium compound in a pharmaceutically acceptable medium compatible with human skin.

The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is Cream II which is a pharmaceutical composition comprising distilled water, neomycin, magnesium sulfate, glycerine, glyceryl dilaurate, glyceryl alcohol, ceteareth-20, isopropyl myristate, imidurea NF, guar gum, methyl pareben, and propyl paraben.

Tetracycline Pharmaceutical Compositions:

The invention also includes a pharmaceutical composition for topical administration for localized chemodenervation comprising pharmaceutically effective amounts of an tetracycline and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of tetracycline and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of tetracycline and a magnesium salt or organic magnesium compound in a pharmaceutically acceptable medium compatible with human skin. The magnesium salt can be magnesium sulfate.

Lincosamide Pharmaceutical Compositions:

The invention also includes a pharmaceutical composition for topical administration for localized chemodenervation comprising pharmaceutically effective amounts of an lincosamide and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of lincosamide and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of lincosamide and a magnesium salt or organic magnesium compound in a pharmaceutically acceptable medium compatible with human skin. The lincosamide can be clindamycin or lincomycin. The magnesium salt can be magnesium sulfate.

Muscle Relaxant Pharmaceutical Compositions:

The invention also includes a pharmaceutical composition for topical administration for localized chemodenervation comprising pharmaceutically effective amounts of an muscle relaxant and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of muscle relaxant and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of muscle relaxant and a magnesium salt or organic magnesium compound in a pharmaceutically acceptable medium compatible with human skin. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. The magnesium salt can be magnesium sulfate.

Plant/Herb Extract Pharmaceutical Compositions:

The invention also includes a pharmaceutical composition for topical administration for localized chemodenervation comprising pharmaceutically effective amounts of an plant/herb extract and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of plant/herb extract and a magnesium salt or organic magnesium compound in a pharmaceutically acceptable medium compatible with human skin. In a yet a further aspect, the invention includes a pharmaceutical composition for topical administration for reducing facial wrinkles comprising pharmaceutically effective amounts of plant/herb extract and a magnesium salt or organic magnesium compound in a pharmaceutically acceptable medium compatible with human skin. The plant extract can be toosendanin, coryneine, banana truck extract or curare. The magnesium salt can be magnesium sulfate.

Combinational Pharmaceutical Compositions:

The compounds of the present invention, e.g., antibiotics (including but not limited to: aminoglycoside antibiotics, tetracyclines, polymyxin B, and lincosamides), magnesium and muscle relaxants, plant extracts (including but not limited to: toosendanin, coryneine, banana truck extract or curare), (also referred to herein as “active compounds”) and derivatives, fragments, analogs and homologs thereof, can be incorporated into pharmaceutical compositions suitable for administration which can then be utilized in combination with other known compounds for localized chemodenervation, treating involuntary facial muscle spasms, or reducing facial wrinkles. Theses compounds are used either to increase the penetration of active ingredients through the skin or to increase the release of active ingredients from the formulation to the skin. These compounds known in the field include, but are not limited to, azone, certain surfactants, DMSO, alcohol, acetone, propyleneglycol and polyethylene glycol. In addition, various drug delivery systems, including liposome and microscopic polymer-based microspares, may be applied to control the rate of drug delivery to the skin or to allow the skin to control the rate of drug absorption. In preferred embodiments, the compounds of the present invention, e.g., antibiotics (including but not limited to: aminoglycoside antibiotics, tetracyclines, polymyxin B, and lincosamides), magnesium and muscle relaxants, and derivatives, fragments, analogs and homologs thereof are utilized in combination with other compounds of the present invention (e.g., aminoglycoside antibiotics in combination with magnesium, polymyxin B in combination with muscle relaxants etc.) for localized chemodenervation, treating involuntary facial muscle spasms, or reducing facial wrinkles. In preferred embodiments, two or more compounds are utilized in combination.

Kits:

The pharmaceutical compositions can be included in a kit, container, pack, or dispenser together with instructions for administration. In one aspect, the invention includes a kit comprising any of the pharmaceutical compositions for localized chemodenervation described herein and instructions for topical administration of the composition. The pharmaceutical composition can be antibiotics (including but not limited to: aminoglycoside antibiotics, tetracyclines, polymyxin B, and lincosamides), magnesium, plant and herb extracts and muscle relaxants. The aminoglycoside antibiotic can be amikacin, gentamicin, kanamycin, neomycin, netilmicin, paromomycin, streptomycin or tobramycin. The lincosamide can be clindamycin or lincomycin. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound. The plant extract can be toosendanin, coryneine, banana truck extract or curare. In a preferred embodiment, the pharmaceutical composition can further comprise a magnesium salt or an organic magnesium compound.

In another aspect, the invention includes a kit comprising any of the pharmaceutical compositions for treating involuntary muscle spasms described herein and instructions for topical administration of the composition. The pharmaceutical composition can be antibiotics (including but not limited to: aminoglycoside antibiotics, tetracyclines, polymyxin B, and lincosamides), magnesium, plant and herb extracts and muscle relaxants. The aminoglycoside antibiotic can be amikacin, gentamicin, kanamycin, neomycin, netilmicin, paromomycin, streptomycin or tobramycin. The lincosamide can be clindamycin or lincomycin. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound. The plant extract can be toosendanin, coryneine, banana truck extract or curare. In a preferred embodiment, the pharmaceutical composition can further comprise a magnesium salt or an organic magnesium compound.

In yet another aspect, the invention includes a kit comprising any of the cosmetic compositions for treating facial wrinkles described herein and instructions for topical administration of the composition. The pharmaceutical composition can be antibiotics (including but not limited to: aminoglycoside antibiotics, tetracyclines, polymyxin B, and lincosamides), magnesium, plant and herb extracts and muscle relaxants. The aminoglycoside antibiotic can be amikacin, gentamicin, kanamycin, neomycin, netilmicin, paromomycin, streptomycin or tobramycin. The lincosamide can be clindamycin or lincomycin. The muscle relaxant can be a non-depolarizing or depolarizing agent. In preferred embodiments, the non-depolarizing agent can be pancuronium, vecuronium, mivacurium or rocuronium and the depolarizing agent can be succinylcholine or decamethonium. In another preferred embodiment, the muscle relaxant is a magnesium salt or an organic magnesium compound. The plant extract can be toosendanin, coryneine, banana truck extract or curare. In a preferred embodiment, the pharmaceutical composition can further comprise a magnesium salt or an organic magnesium compound.

The invention will be further described in the following examples, which do not limit the scope of the invention described in the claims.

### EXAMPLES

### Example 1

### Topical Use of Cream I for Anti-Wrinkle Effect

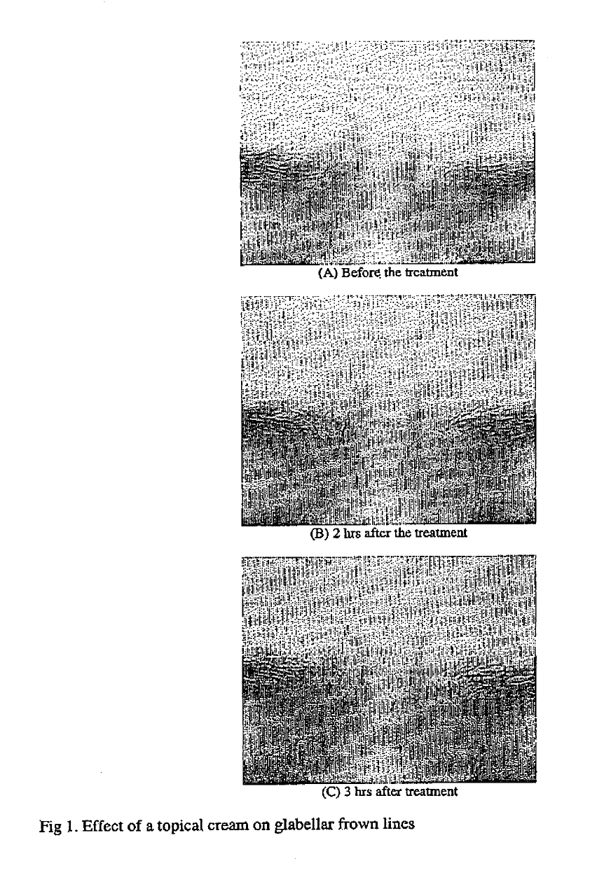

Cream I was applied to the skin area around facial frowns. Pictures were taken pre-treatment and post-treatment on hourly basis. As shown in FIG. 1, after applying the cream, facial frown lines were significantly reduced just two hours after the treatment and kept improving by three hours. In addition, more smooth skin and relaxed muscle were observed after application (FIG. 1). Compared to Botox injection, topical use of the cream delivered its effect much faster and did not cause any numbness or stiffness.

### Example 2

### Topical Use of Cream I for Treating Synkinesis

Cream I was tested in a patient with synkinesis. The patient, a 44 years old male, had surgery for acoustic neuroma two years ago. During the recovery from facial paralysis, he developed minor synkinesis. When he tried to pucker and stretch his mouth, involuntary muscle contraction under his right eye was observed (left eye is perfectly normal).

Cream I was applied to the area under the patient's right eye. In 10-15 minutes, the involuntary muscle contraction was significantly reduced. By 30 minutes, the synkinesis was almost completely gone. The effect lasted 5-6 hours. After that, the synkinesis slowly returned.

The therapeutic effect of this cream results from the presence of polymyxin B, which demonstrates a dose-response property: while a control cream, containing no polymyxin B, did not show any activity, the inhibition of synkinesis became more obvious as the concentration of polymyxin B increased (4,000, 10,000, 20,000 and 40,000 U/g were tested).

On the other hand, polymyxin B alone (an over-the-counter drug containing 10,000 U/g polymyxin B was tested) did not show any inhibition of synkinesis, thereby suggesting the importance of magnesium, another neuromuscular blocking agent in the formulation.

### Example 3

### Anti-Wrinkle Effect of Neomycin

In order to demonstrate that other compounds of the instant invention may work in a similar fashion as the Cream I, Cream II was formulated using the aminoglycoside neomycin and was applied to frown lines. After one hour, frown lines were visibly reduced. The cream can be applied again after 0.5-1 hour to achieve a better result.

### Example 4

### Preparation Of Plant Extracts With Neuromuscular Blocking Activity

Plants and/or herbs were ground and extracted with distilled water. After boiling for 10 minutes and cooling down to room temperature overnight, the extracts were separated from any insoluble fraction by centrifugation at 5,000 rpm for five minutes. The extracts were then concentrated on a rotary evaporator and stored at −20° C. for testing in an animal model or human volunteers.

| | | Dry | | Final |
|---|---|---|---|---|
| | Active | weight | Extraction | volume |
| Plant/herb | ingredient | (g) | volume (ml) | (ml) |
| Fructus Meliae | Toosendanin | 33.8 | 250 | 18 |
| Aconite Root | coryneine | 22 | 150 | 16 |
| Cortex Meliae | Toosendanin | 19 | 150 | 12 |
| Black Cohosh | ciminifuga | 30 | 440 | 13 |
| | racemose | | | |
| Passion Flower | passiflora | 36 | 570 | 25 |
| | incarnata | | | |
| Kava | piper | 47 | 650 | 26 |
| | methysticum | | | |

### Example 5

### Animal Model

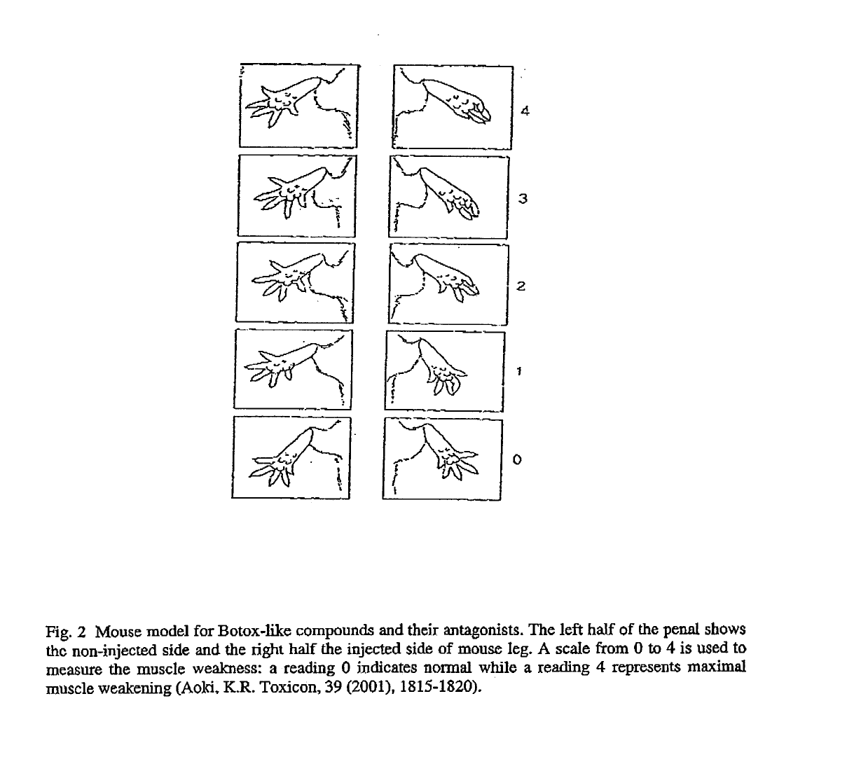

Based on a procedure used by Allergan for analysis of Botox product (Aoki, Toxicon 39: 1815-1820, 2001), a mouse model was established to examine the neuromuscular blockade potential of various compounds described above. After a compound was topically applied to the gastrocnemius muscle of mouse legs, its effect could be semi-quantitatively measured by briefly suspending the tail to elicit a characteristic startle response in which the mouse extends its hind limbs and abducts its hind digits (FIG. 2).

For example, the antagonistic effect of 3,4-diaminopyridine (DAP) was tested on Botox by topical administration. DAP belongs to a group of compounds that act on nerve membranes to promote influx of calcium, which in turn greatly promotes efflux of acetylcholine. In a group of mice (n=7), average reading after Botox treatment is 2.5 (in a scale from 0 to 4, 0 being normal and 4 being most severe). After topically applying DAP on the legs, the reading was recorded as 1.5 at 1 hour and continued to drop to 0.75 at 4 hour (FIG. 3).

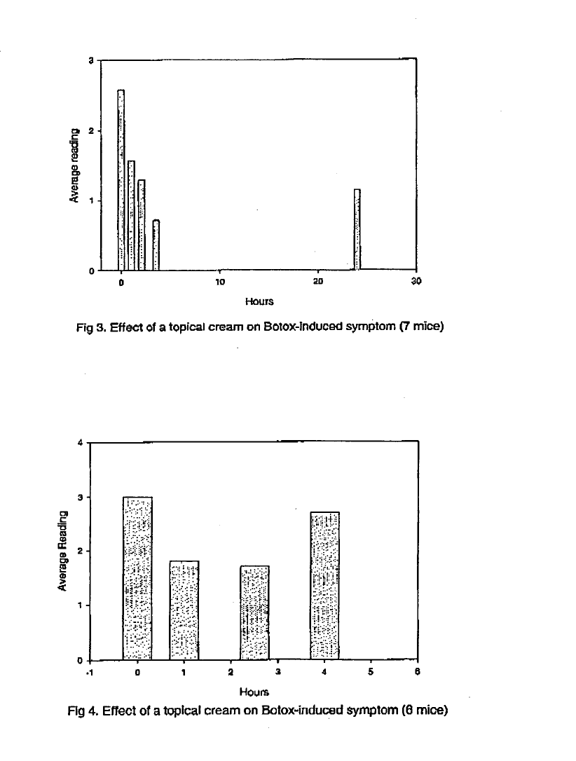

In a separate experiment (mouse number=6), rapid response to topical administration of DAP was observed, although the reading went up after 4 hours of the treatment (FIG. 4). This study indicates that this mouse model can be effectively used to test and screen compounds, by topical administration, for activities of modulating neuromuscular transmission.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for localized chemodenervation comprising topically administering a pharmaceutical composition comprising a pharmaceutically effective amount of an antibiotic and a pharmaceutically acceptable carrier.

2. A method for localized chemodenervation comprising topically administering a pharmaceutical composition comprising a pharmaceutically effective amount of aminoglycoside antibiotic and a pharmaceutically acceptable carrier.

3. The method of claim 2, wherein the aminoglycoside antibiotic is selected from the group consisting of amikacin, gentamicin, kanamycin, neomycin, netilmicin, paromomycin, streptomycin and tobramycin.

4. The method of claim 2, wherein the pharmaceutical composition further comprises a magnesium salt or an organic magnesium compound.

5. A method for localized chemodenervation comprising topically administering a pharmaceutical composition comprising a pharmaceutically effective amount of polymyxin B and a pharmaceutically acceptable carrier.

6. The method of claim 5, wherein the pharmaceutical composition further comprises a magnesium salt or an organic magnesium compound.

7. A method for localized chemodenervation comprising topically administering a pharmaceutical composition for comprising a pharmaceutically effective amount of tetracycline and a pharmaceutically acceptable carrier.

8. A method for localized chemodenervation comprising topically administering a pharmaceutical composition comprising a pharmaceutically effective amount of lincosamide and a pharmaceutically acceptable carrier.

9. The method of claim 8 wherein the lincosamide is clindamycin or lincomycin.

10. A method for localized chemodenervation comprising topically administering a pharmaceutical composition comprising a pharmaceutically effective amount of a muscle relaxant and pharmaceutically acceptable carrier.

11. The method of claim 10 wherein the muscle relaxant is a non-depolarizing agent.

12. The method of claim 11 wherein the non-depolarizing agent is pancuronium, vecuronium, mivacurium or rocuronium.

13. The method of claim 10 wherein the muscle relaxant is a depolarizing agent.

14. The method of claim 13 wherein the depolarizing agent is succinylcholine or decamethonium.

15. The method of claim 10 wherein the muscle relaxant is a magnesium salt or an organic magnesium compound.

16. A method for localized chemodenervation comprising topically administering a pharmaceutical composition comprising a pharmaceutically effective amount of one or more compounds selected from the group consisting of a magnesium salt, an organic magnesium compound or a plant extract, and a pharmaceutically acceptable carrier.

17. The method of claim 16, wherein the plant extract is toosendanin, coryneine, banana truck extract or curare.

18. A method for treating involuntary facial muscle spasms comprising topically administering a pharmaceutical composition comprising a pharmaceutically effective amount of an antibiotic and a pharmaceutically acceptable carrier.

19. A method for treating involuntary facial muscle spasms comprising topically administering a pharmaceutical composition comprising a pharmaceutically effective amount of an aminoglycoside antibiotic and a pharmaceutically acceptable carrier.

20. The method of claim 19, wherein the aminoglycoside antibiotic is selected from the group consisting of amikacin, gentamicin, kanamycin, neomycin, netilmicin, paromomycin, streptomycin and tobramycin.

21. The method of claim 19, wherein the pharmaceutical composition further comprises a magnesium salt or an organic magnesium compound.

22. A method for treating involuntary facial muscle spasms comprising topically administering a pharmaceutical composition comprising a pharmaceutically effective amount of polymyxin B and a pharmaceutically acceptable carrier.

23. The method of claim 22, wherein the pharmaceutical composition further comprises a magnesium salt or an organic magnesium compound.

24. A method for treating involuntary facial muscle spasms comprising topically administering a pharmaceutical composition comprising a pharmaceutically effective amount of tetracycline and a pharmaceutically acceptable carrier.

25. A method for treating involuntary facial muscle spasms comprising topically administering a pharmaceutical composition comprising a pharmaceutically effective amount of a lincosamide and a pharmaceutically acceptable carrier.

26. The method of claim 25 wherein the lincosamide is clindamycin or lincomycin.

27. A method for treating involuntary facial muscle spasms comprising topically administering a pharmaceutical composition comprising a pharmaceutically effective amount of a muscle relaxant and a pharmaceutically acceptable carrier.

28. The method of claim 27 wherein the muscle relaxant is a non-depolarizing agent.

29. The method of claim 28 wherein the non-depolarizing agent is pancuronium, vecuronium, mivacurium or rocuronium.

30. The method of claim 27 wherein the muscle relaxant is a depolarizing agent.

31. The method of claim 30 wherein the depolarizing agent is succinylcholine or decamethonium.

32. The method of claim 27 wherein the muscle relaxant is a magnesium salt or an organic magnesium compound.

33. A method for treating involuntary facial muscle spasms comprising topically administering a pharmaceutical composition comprising a pharmaceutically effective amount of one or more compounds selected from the group consisting of a magnesium salt, an organic magnesium compound or a plant extract, and a pharmaceutically acceptable carrier.

34. The method of claim 33, wherein the plant extract is toosendanin, coryneine, banana truck extract or curare.

35. The method of claim 18, 19, 22, 24, 25, 27 or 33 wherein the involuntary facial muscle spasms are caused by synkinesis, ocular disorders, dystonia, hemifacial spasm, or blepharospasm.

36. A method for reducing facial wrinkles comprising topically administering a pharmaceutical composition comprising a pharmaceutically effective amount of an antibiotic and a pharmaceutically acceptable carrier.

37. A method for reducing facial wrinkles comprising topically administering a pharmaceutical composition comprising a pharmaceutically effective amount of an aminoglycoside antibiotic and a pharmaceutically acceptable carrier.

38. The method of claim 37, wherein the aminoglycoside antibiotic is selected from the group consisting of amikacin, gentamicin, kanamycin, neomycin, netilmicin, paromomycin, streptomycin and tobramycin.

39. The method of claim 37, wherein the pharmaceutical composition further comprises a magnesium salt or an organic magnesium compound.

40. A method for reducing facial wrinkles comprising topically administering a pharmaceutical composition comprising a pharmaceutically effective amount of polymyxin B and a pharmaceutically acceptable carrier.

41. The method of claim 40, wherein the pharmaceutical composition further comprises a magnesium salt or an organic magnesium compound.

42. A method for reducing facial wrinkles comprising topically administering a pharmaceutical composition comprising a pharmaceutically effective amount of tetracycline and a pharmaceutically acceptable carrier.

43. A method for reducing facial wrinkles comprising topically administering a pharmaceutical composition comprising a pharmaceutically effective amount of a lincosamide and a pharmaceutically acceptable carrier.

44. The method of claim 43 wherein the lincosamide is clindamycin or lincomycin.

45. A method for reducing facial wrinkles comprising topically administering a pharmaceutical composition comprising a pharmaceutically effective amount of a muscle relaxant and a pharmaceutically acceptable carrier.

46. The method of claim 45 wherein the muscle relaxant is a non-depolarizing agent.

47. The method of claim 46 wherein the non-depolarizing agent is pancuronium, vecuronium, mivacurium or rocuronium.

48. The method of claim 45 wherein the muscle relaxant is a depolarizing agent.

49. The method of claim 49 wherein the depolarizing agent is succinylcholine or decamethonium.

50. The method of claim 45 wherein the muscle relaxant is a magnesium salt or an organic magnesium compound.

51. A method for reducing facial wrinkles comprising topically administering a pharmaceutical composition comprising a pharmaceutically effective amount of one or more compounds selected from the group consisting of a magnesium salt, an organic magnesium compound or a plant extract, and a pharmaceutically acceptable carrier.

52. The method of claim 51, wherein the plant extract is toosendanin, coryneine, banana truck extract or curare.

53. A pharmaceutical composition for topical administration for localized chemodenervation comprising pharmaceutically effective amounts of
polymyxin B and
a magnesium salt or organic magnesium compound in a pharmaceutically acceptable medium compatible with human skin.

54. The pharmaceutical composition of claim 53 wherein the amount of polymyxin B is between about 800 U/g and about 80,000 U/g.

55. The pharmaceutical composition of claim 53 wherein the magnesium salt is magnesium sulfate.

56. The pharmaceutical composition of claim 55 wherein the amount of magnesium sulfate is between about 2 mg/g and about 200 mg/g.

57. A pharmaceutical composition for topical administration for localized chemodenervation comprising pharmaceutically effective amounts of
an aminoglycoside antibiotic and
a magnesium salt or organic magnesium compound in a pharmaceutically acceptable medium compatible with human skin.

58. The pharmaceutical composition of claim 57 wherein the aminoglycoside antibiotic is selected from the group consisting of amikacin, gentamicin, kanamycin, neomycin, netilmicin, paromomycin, streptomycin and tobramycin.

59. The pharmaceutical composition of claim 58 wherein the amount of neomycin is between about 2.5 mg/g and about 200 mg/g.

60. The pharmaceutical composition of claim 57 wherein the magnesium salt is magnesium sulfate.

61. The pharmaceutical composition of claim 60 wherein the amount of magnesium sulfate is between about 2 mg/g and about 200 mg/g.

62. A pharmaceutical composition for topical administration for localized chemodenervation comprising a pharmaceutically effective amount of tetracycline and in a pharmaceutically acceptable medium compatible with human skin.

63. The pharmaceutical composition of claim 62 further comprising a magnesium salt or organic magnesium compound.

64. The pharmaceutical composition of claim 63 wherein the magnesium salt is magnesium sulfate.

65. A pharmaceutical composition for topical administration for localized chemodenervation comprising a pharmaceutically effective amount of lincosamide in a pharmaceutically acceptable medium compatible with human skin.

66. The pharmaceutical composition of claim 65 wherein the lincosamide is clindamycin or lincomycin.

67. The pharmaceutical composition of claim 65 further comprising a magnesium salt or organic magnesium compound.

68. The pharmaceutical composition of claim 67 wherein the magnesium salt is magnesium sulfate.

69. A pharmaceutical composition for topical administration for localized chemodenervation comprising pharmaceutically effective amount of a muscle relaxant in a pharmaceutically acceptable medium compatible with human skin.

70. The pharmaceutical composition of claim 69 wherein the muscle relaxant is a non-depolarizing agent.

71. The pharmaceutical composition of claim 70 wherein the non-depolarizing agent is pancuronium, vecuronium, mivacurium or rocuronium.

72. The pharmaceutical composition of claim 69 wherein the muscle relaxant is a depolarizing agent.

73. The pharmaceutical composition of claim 72 wherein the depolarizing agent is succinylcholine or decamethonium.

74. The pharmaceutical composition of claim 69 wherein the muscle relaxant is a magnesium salt or an organic magnesium compound.

75. The pharmaceutical composition of claim 69 further comprising a magnesium salt or organic magnesium compound.

76. The pharmaceutical composition of claim 75 wherein the magnesium salt is magnesium sulfate.

77. A kit comprising the composition of claim 53, 57, 62, 65 or 69 and instructions on how to administer said composition for localized chemodenervation.

78. A pharmaceutical composition for topical administration for treating involuntary facial muscle spasms comprising pharmaceutically effective amounts of
polymyxin B and
a magnesium salt or organic magnesium compound in a pharmaceutically acceptable medium compatible with human skin.

79. The pharmaceutical composition of claim 78 wherein the amount of polymyxin B is between about 800 U/g and about 80,000 U/g.

80. The pharmaceutical composition of claim 78 wherein the magnesium salt is magnesium sulfate.

81. The pharmaceutical composition of claim 80 wherein the amount of magnesium sulfate is between about 2 mg/g and about 200 mg/g.

82. A pharmaceutical composition for topical administration for treating involuntary facial muscle spasms comprising pharmaceutically effective amounts of
an aminoglycoside antibiotic and
a magnesium salt or organic magnesium compound in a pharmaceutically acceptable medium compatible with human skin.

83. The pharmaceutical composition of claim 82 wherein the aminoglycoside antibiotic is selected from the group consisting of amikacin, gentamicin, kanamycin, neomycin, netilmicin, paromomycin, streptomycin and tobramycin.

84. The pharmaceutical composition of claim 83 wherein the amount of neomycin is between about 2.5 mg/g and about 200 mg/g.

85. The pharmaceutical composition of claim 82 wherein the magnesium salt is magnesium sulfate.

86. The pharmaceutical composition of claim 85 wherein the amount of magnesium sulfate is between about 2 mg/g and about 200 mg/g.

87. A pharmaceutical composition for topical administration for treating involuntary facial muscle spasms comprising a pharmaceutically effective amount of tetracycline in a pharmaceutically acceptable medium compatible with human skin.

88. The pharmaceutical composition of claim 87 further comprising a magnesium salt or organic magnesium compound.

89. The pharmaceutical composition of claim 88 wherein the magnesium salt is magnesium sulfate.

90. A pharmaceutical composition for topical administration for treating involuntary facial muscle spasms comprising a pharmaceutically effective amount of lincosamide in a pharmaceutically acceptable medium compatible with human skin.

91. The pharmaceutical composition of claim 90 wherein the lincosamide is clindamycin or lincomycin.

92. The pharmaceutical composition of claim 90 further comprising a magnesium salt or organic magnesium compound.

93. The pharmaceutical composition of claim 92 wherein the magnesium salt is magnesium sulfate.

94. A pharmaceutical composition for topical administration for treating involuntary facial muscle spasms comprising a pharmaceutically effective amount of a muscle relaxant in a pharmaceutically acceptable medium compatible with human skin.

95. The pharmaceutical composition of claim 94 wherein the muscle relaxant is a non-depolarizing agent.

96. The pharmaceutical composition of claim 95 wherein the non-depolarizing agent is pancuronium, vecuronium, mivacurium or rocuronium.

97. The pharmaceutical composition of claim 94 wherein the muscle relaxant is a depolarizing agent.

98. The pharmaceutical composition of claim 97 wherein the depolarizing agent is succinylcholine or decamethonium.

99. The pharmaceutical composition of claim 94 wherein the muscle relaxant is a magnesium salt or an organic magnesium compound.

100. The pharmaceutical composition of claim 94 further comprising a magnesium salt or organic magnesium compound.

101. The pharmaceutical composition of claim 100 wherein the magnesium salt is magnesium sulfate.

102. A kit comprising the composition of claim 78, 82, 87, 90 or 94 and instructions on how to administer said composition to treat involuntary facial muscle spasms.

103. A pharmaceutical composition for topical administration for reducing facial wrinkles comprising effective amounts of
polymyxin B and
a magnesium salt or organic magnesium compound in a medium compatible with human skin.

104. The pharmaceutical composition of claim 103 wherein the amount of polymyxin B is between about 800 U/g and about 80,000 U/g.

105. The pharmaceutical composition of claim 103 wherein the magnesium salt is magnesium sulfate.

106. The pharmaceutical composition of claim 105 wherein the amount of magnesium sulfate is between about 2 mg/g and about 200 mg/g.

107. A pharmaceutical composition for topical administration for reducing facial wrinkles comprising effective amounts of
an aminoglycoside antibiotic and
a magnesium salt or organic magnesium compound in a medium compatible with human skin.

108. The pharmaceutical composition of claim 107 wherein the aminoglycoside antibiotic is selected from the group consisting of amikacin, gentamicin, kanamycin, neomycin, netilmicin, paromomycin, streptomycin and tobramycin.

109. The pharmaceutical composition of claim 108 wherein the amount of neomycin is between about 2.5 mg/g and about 200 mg/g.

110. The pharmaceutical composition of claim 107 wherein the magnesium salt is magnesium sulfate.

111. The pharmaceutical composition of claim 110 wherein the amount of magnesium sulfate is between about 2 mg/g and about 200 mg/g.

112. A pharmaceutical composition for topical administration for reducing facial wrinkles comprising a pharmaceutically effective amount of tetracycline in a pharmaceutically acceptable medium compatible with human skin.

113. The pharmaceutical composition of claim 112 further comprising a magnesium salt or organic magnesium compound.

114. The pharmaceutical composition of claim 113 wherein the magnesium salt is magnesium sulfate.

115. A pharmaceutical composition for topical administration for reducing facial wrinkles comprising a pharmaceutically effective amount of lincosamide in a pharmaceutically acceptable medium compatible with human skin.

116. The pharmaceutical composition of claim 115 wherein the lincosamide is clindamycin or lincomycin.

117. The pharmaceutical composition of claim 115 further comprising a magnesium salt or organic magnesium compound.

118. The pharmaceutical composition of claim 117 wherein the magnesium salt is magnesium sulfate.

119. A pharmaceutical composition for topical administration for reducing facial wrinkles comprising a pharmaceutically effective amount of a muscle relaxant in a pharmaceutically acceptable medium compatible with human skin.

120. The pharmaceutical composition of claim 119 wherein the muscle relaxant is a non-depolarizing agent.

121. The pharmaceutical composition of claim 120 wherein the non-depolarizing agent is pancuronium, vecuronium, mivacurium or rocuronium.

122. The pharmaceutical composition of claim 119 wherein the muscle relaxant is a depolarizing agent.

123. The pharmaceutical composition of claim 122 wherein the depolarizing agent is succinylcholine or decamethonium.

124. The pharmaceutical composition of claim 119 wherein the muscle relaxant is a magnesium salt or an organic magnesium compound.

125. The pharmaceutical composition of claim 119 further comprising a magnesium salt or organic magnesium compound.

126. The pharmaceutical composition of claim 125 wherein the magnesium salt is magnesium sulfate.

127. A kit comprising the composition of claim 103, 107, 112, 115 or 119 and instructions on how to administer said composition to reduce facial wrinkles.
